(19) United States
(12) Patent Application Publication
(10) Pub. No.: US 2015/0152445 A1
(43) Pub. Date: Jun. 4, 2015

(54) MICROORGANISMS AND METHODS FOR THE PRODUCTION OF KETONES

(71) Applicant: LanzaTech New Zealand Limited, Skokie, IL (US)

(72) Inventors: Michael Koepke, Skokie, IL (US); Bakir Al-Sinawi, Sydney (AU); Rasmus Overgaard Jensen, Skokie, IL (US); Alexander Paul Mueller, Skokie, IL (US); Ryan Edward Hill, Otago (NZ)

(21) Appl. No.: 14/559,881

(22) Filed: Dec. 3, 2014

Related U.S. Application Data
(60) Provisional application No. 61/911,449, filed on Dec. 3, 2013.

(51) Int. Cl. C12P 7/28; C12N 9/88; C12P 7/26; C12N 9/10

(52) U.S. Cl. CPC C12P 7/28; C12N 9/1029; C12N 9/13; C12N 9/88; C12P 7/26; C12Y 208/03; C12Y 401/01004; C12Y 402/01028

(57) ABSTRACT

The invention provides a recombinant, acetogenic, carboxydotrophic bacterium that lacks secondary alcohol dehydrogenase or comprises an inactivated secondary alcohol dehydrogenase. The inactivated secondary alcohol dehydrogenase may be encoded by a secondary alcohol dehydrogenase gene comprising an inactivating mutation that reduces the ability of the bacterium to convert acetone to isopropanol and to convert methyl ethyl ketone to 2-butanol. Since a bacterium that lacks secondary alcohol dehydrogenase or comprises an inactivated secondary alcohol dehydrogenase accumulates carbonyl-containing compounds, the invention also provides a method of producing carbonyl-containing compounds, such as acetone and methyl ethyl ketone.

### CROSS REFERENCE TO RELATED APPLICATIONS

This application claims the benefit of U.S. Provisional Patent Application No. 61/911,449 filed Dec. 3, 2013, the entirety of which is incorporated herein by reference.

### SEQUENCE LISTING

This application includes a nucleotide/amino acid sequence listing submitted concurrently herewith and identified as follows: 7,617 byte ASCII (text) file named “LT101US1_ST25.txt” created on Dec. 1, 2014, the entirety of which is incorporated herein by reference.

### TECHNICAL FIELD OF THE INVENTION

This invention relates to industrial fermentation. In particular, it relates to the use of microorganisms to produce industrially useful solvents, such as ketones.

### BACKGROUND OF THE INVENTION

Acetogenic, carboxydotrophic bacteria, such as Clostridium autoethanogenum, Clostridium ljungdahlii, and Clostridium ragsdalei convert acetone to isopropanol and methyl ethyl ketone (MEK) to 2-butanol. Accordingly, these bacteria produce alcohols, such as isopropanol and 2-butanol, at the expense of ketones, such as acetone and MEK.

However, ketones themselves are important commercial products. Acetone is an industrial solvent and precursor for methyl methacrylate (MMA) and polymethyl methacrylate (PMMA), which have a combined market value of about $10 billion USD/year. Acetone is also a precursor for isobutylene (van Leeuwen, Appl Microbiol Biotechnol, 93: 1377-1387, 2012). The market value of isobutylene is also substantial, at about $24 billion USD/year. MEK is an important component of paints and inks, with a global market value of about $2 billion USD/year, growing at about 1.9% per year. MEK is also a precursor for 1,3-butadiene, which has a market value exceeding $19 billion USD/year, growing at about 2.7% per year. Furthermore, acetone is a precursor for production of jet fuels (Anbarasan, Nature, 491: 235-239, 2012).

Accordingly, there remains a need for improved methods of producing industrially useful solvents and synthetic precursors, such as acetone and methyl ethyl ketone (MEK).

### SUMMARY OF THE INVENTION

The invention provides a recombinant acetogenic, carboxydotrophic bacterium that lacks a secondary alcohol dehydrogenase or comprises an inactivated secondary alcohol dehydrogenase. Preferably, the bacterium is a member of the genus Clostridium, such as a bacterium derived from Clostridium autoethanogenum, Clostridium ljungdahlii, or Clostridium ragsdalei. In a particular embodiment, the bacterium is derived from Clostridium autoethanogenum deposited under DSMZ Accession No. DSM23693.

The inactivated secondary alcohol dehydrogenase may be derived from an enzyme having secondary alcohol dehydrogenase or primary-secondary alcohol dehydrogenase activity. The inactivated secondary alcohol dehydrogenase may be encoded by a secondary alcohol dehydrogenase gene comprising an inactivating mutation. The secondary alcohol dehydrogenase gene comprising the inactivating mutation may be derived from a gene encoding an enzyme having secondary alcohol dehydrogenase or primary-secondary alcohol dehydrogenase activity. In one embodiment, the inactivated secondary alcohol dehydrogenase is derived from the amino acid sequence of SEQ ID NO: 1. In another embodiment, the secondary alcohol dehydrogenase gene comprising the inactivating mutation is derived from the nucleic acid sequence of SEQ ID NO: 2 or SEQ ID NO: 3. In particular, the inactivating mutation may be an insertion.

The bacterium may further comprise exogenous genes encoding one or more enzymes, such as thiolase, coA-transferase, or acetoacetate decarboxylase. Furthermore, the bacterium may comprise an exogenous gene encoding propanediol dehydratase that converts meso-2,3-butanediol to MEK, such as Klebsiella oxtoca propanediol dehydratase.

The presence of an inactivated secondary alcohol dehydrogenase results in the accumulation of ketones. Accordingly, the bacterium may accumulate or produce ketones, such as acetone or MEK.

The invention further provides methods of producing ketones, such as acetone or MEK, by culturing the bacterium comprising an inactivated secondary alcohol dehydrogenase encoded by a secondary alcohol dehydrogenase gene comprising an inactivating mutation, whereby the bacterium produces one or more of acetone and MEK. The method may further comprise recovering the acetone or MEK from the culture.

### BRIEF DESCRIPTION OF THE DRAWINGS

FIG. 1. is a table showing the kinetics of C. autoethanogenum alcohol dehydrogenases on various substrates.

FIG. 2. is a gene map showing intron target sites (211s, 287a, 388a, 400s, 433s, and 552a) and primer binding sites (156F and 939R) on a secondary alcohol dehydrogenase gene.

FIGS. 3A-3C are gel images confirming group II intron insertions.

FIG. 4 is a graph showing the conversion (or lack thereof) of acetone to isopropanol by LZ1561 and mutant C. autoethanogenum with inactivated secondary alcohol dehydrogenase (287a).

FIGS. 5A-5C are graphs showing growth as measured by optical density (FIG. 5A), change in acetone (FIG. 5B), and change in isopropanol (FIG. 5C) over 4 days of growth of a culture of LZ1561 compared to cultures of mutant C. autoethanogenum with inactivated secondary alcohol dehydrogenase (433s, 388a).

FIGS. 6A and 6B are graphs showing complete conversion of acetone to isopropanol at high concentrations and rates when fed into a stable continuous culture of LZ1561 with CO-containing steel mill gas as substrate. Feed concentrations are indicated below each graph.

FIG. 7 is a graph showing acetate (triangles), ethanol (diamonds), and biomass (squares) concentrations on days 11-14 of a fermentation of mutant C. autoethanogenum with inactivated secondary alcohol dehydrogenase. Acetone was fed into the fermenter on day 11 and MEK on day 12.

FIG. 8 is a graph showing acetone (triangles) and isopropanol (diamonds) concentrations detected by HPLC, and a theoretical washout curve for acetone at dilution rate 1.6 (dotted line). The discrepancy is due to gas stripping of acetone.

FIG. 9. is a graph showing the conversion (or lack thereof) of MEK to 2-butanol by LZ1561 and mutant C. autoethanogenum with inactivated secondary alcohol dehydrogenase (287a).

FIG. 10. is a graph showing MEK (triangles) and 2-butanol (diamonds) detected by HPLC, and theoretical washout curve for MEK at dilution rate 1.6 (dotted line). The discrepancy is due to gas stripping of MEK.

FIG. 11. is a graph showing the production of acetone from CO in continuous fermentation in mutant C. autoethanogenum with inactivated secondary alcohol dehydrogenase.

### DETAILED DESCRIPTION OF THE INVENTION

Secondary alcohol dehydrogenases convert carbonyl-containing compounds to alcohols. Accordingly, bacteria containing a secondary alcohol dehydrogenase are capable of converting carbonyl-containing compounds, such as acetone and MEK, to alcohols, such as isopropanol and 2-butanol, respectively. The invention provides a recombinant acetogenic, carboxydotrophic bacterium that lacks a secondary alcohol dehydrogenase or comprises an inactivated secondary alcohol dehydrogenase. In particular, the inactivated secondary alcohol dehydrogenase may be encoded by a secondary alcohol dehydrogenase gene comprising an inactivating mutation, wherein the inactivating mutation reduces the ability of the bacterium to convert acetone to isopropanol and to convert MEK to 2-butanol. Since this inactivating mutation results in the accumulation of carbonyl-containing compounds, the invention also provides a method of producing carbonyl-containing compounds such as acetone and MEK.

A carbonyl is a functional group composed of a carbon atom double-bonded to an oxygen atom, such as an aldehyde or a ketone. Carbonyl-containing compounds include, for example, acetoin, acetone, methyl ethyl ketone (MEK) (i.e., 2-butanone), and acetaldehyde.

An alcohol is an organic compound in which a hydroxyl functional group is bound to a saturated carbon atom. Alcohols include, for example, isopropanol and 2-butanol.

Alcohol dehydrogenases (ADH) are a group of enzymes that catalyse the interconversion between aldehydes or ketones (such as acetone) and alcohols (such as isopropanol). Primary alcohol dehydrogenases are capable of converting aldehydes to primary alcohols, or vice versa, and secondary alcohol dehydrogenases are capable of converting ketones to secondary alcohols, or vice versa. Additionally, a number of alcohol dehydrogenases are capable of converting aldehydes to primary alcohols, or vice versa, and ketones to secondary alcohols, or vice versa. These alcohol dehydrogenases may be referred to as primary-secondary alcohol dehydrogenases, since they demonstrate activity of both primary alcohol dehydrogenases and secondary alcohol dehydrogenases. As used herein, the terms “alcohol dehydrogenase” and “secondary alcohol dehydrogenase” encompass both secondary alcohol dehydrogenases and primary-secondary alcohol dehydrogenases.

The bacterium may lack a secondary alcohol dehydrogenase or may be genetically engineered to not express a secondary alcohol dehydrogenase. In particular, the bacterium may be a bacterium derived from C. autoethanogenum, C. ljungdahlii, or C. ragsdalei (which are the only species of acetogenic bacteria known to have a secondary alcohol dehydrogenase) that lacks a secondary alcohol dehydrogenase.

An inactivating mutation is any mutation that reduces, substantially eliminates, or completely eliminates the activity of an enzyme, for example, a secondary alcohol dehydrogenase or primary-secondary alcohol dehydrogenase. The inactivating mutation may be an insertion, deletion, substitution, or nonsense mutation, or any other type of mutation that reduces or eliminates the activity of the enzyme. The inactivating mutation may reduce the enzyme activity of the bacterium by at least 10%, at least 20%, at least 30%, at least 40%, at least 50%, at least 60%, at least 70%, at least 80%, at least 90%, at least 95%, or at least 99%. The enzyme activity may be reduced or eliminated by reducing the function of the enzyme or by reducing the amount of enzyme. Methods of introducing inactivating mutations are well known in the art. For example, the inactivating mutation may be made by chemical mutagenesis, transposon mutagenesis, viral mutagenesis, or in vitro mutagenesis.

With respect to nucleic acid sequences and amino acid sequences, the term “derived” refers to the modification of a parent or wild-type nucleic acid sequence or amino acid sequence. For example, a secondary alcohol dehydrogenase nucleic acid sequence or amino acid sequence comprising an inactivating mutation may be derived from a parent or wild-type nucleic acid sequence or amino acid sequence, respectively, that does not comprise the inactivating mutation. In a preferred embodiment, the inactivated secondary alcohol dehydrogenase is derived from a wild-type secondary alcohol dehydrogenase comprising the amino acid sequence of SEQ ID NO: 1. A gene encoding the inactivated secondary alcohol dehydrogenase may be derived from a nucleic acid sequence comprising SEQ ID NO: 2 or SEQ ID NO: 3.

The invention may be practiced using nucleic acids whose sequence varies from the sequences specifically exemplified herein, provided the nucleic acids perform substantially the same function. For nucleic acid sequences that encode a protein or peptide, this means that the encoded protein or peptide performs substantially the same function. For nucleic acid sequences of promoters, variant sequences will have the ability to promote expression of one or more genes. Such nucleic acids may be referred to as “functionally equivalent variants.” By way of example, functionally equivalent variants of a nucleic acid include allelic variants, fragments of a gene, polymorphisms, and the like. Homologous genes from other microorganisms are examples of functionally equivalent variants of the sequences specifically exemplified herein. These include homologous genes in species such as C. autoethanogenum, C. ljungdahlii, C. ragsdalei, or C. novyi, for which sequence information is publicly available on websites such as GenBank or NCBI. The phrase “functionally equivalent variants” also includes nucleic acids whose sequence varies as a result of codon optimization for a particular organism. Functionally equivalent variants of a nucleic acid will preferably have at least approximately 70%, at least approximately 80%, at least approximately 85%, at least approximately 90%, at least approximately 95%, or greater nucleic acid sequence identity with the nucleic acid identified.

Additionally, the invention may be practiced using enzymes or protein whose sequence varies from the sequences specifically exemplified herein, provided the enzymes or proteins perform substantially the same function. These variants may be referred to as “functionally equivalent variants.” A functionally equivalent variant of a protein or a peptide includes those proteins or peptides that share at least 40%, at least 50%, at least 60%, at least 70%, at least 75%, at least 80%, at least 85%, at least 90%, at least 95%, or greater amino acid identity with the protein or peptide identified. Such variants include fragments of a protein or peptide wherein the fragment comprises a truncated form of the polypeptide wherein deletions may be from 1 to 5, to 10, to 15, to 20, to 25 amino acids. The deletions may extend from residue 1 through 25 at either terminus of the polypeptide, may be of any length within the region, or may be at an internal location or a specific domain of the protein conferring a specific catalytic function and activity or binding of substrate or co-factors. Functionally equivalent variants of the specific enzymes or proteins also include polypeptides expressed by homologous genes in other species of bacteria, for example, as exemplified in the previous paragraph.

The nucleic acid sequences and amino acid sequences of the present invention may be exogenous or endogenous. In one embodiment, exogenous nucleic acids are introduced to express a gene not natively produced by the microorganism or to overexpress a gene natively produced by a microorganism. Additionally, the exogenous nucleic acid may increase the copy number of a gene, introduce a strong or constitutive promoter, or introduce a regulatory element. The exogenous nucleic acid may integrate into the genome of the microorganism or may remain in an extra-chromosomal state.

Recombinant nucleic acids, proteins or microorganisms contain portions of different individuals, different species, or different genera that have been joined together. Typically this is done using techniques of recombinant DNA, such that a composite nucleic acid is formed. The composite nucleic acid can be used to make a composite protein, for example. It can be used to make a fusion protein. It can be used to transform a microbe which maintains and replicates the composite nucleic acid and optionally expresses a protein, optionally a composite protein. The bacterium of the present invention is recombinant, i.e., non-wild-type.

Stereospecific enzymes differentially recognize enantiomers and catalyze different reactions with the enantiomers. Thus only one enantiomer may react, or each enantiomer may yield a distinct product.

“Attenuated expression” refers to the expression of a nucleic acid or protein that is decreased relative to the expression in a parental microorganism. Attenuated expression also includes “zero” expression which refers to the nucleic acid or protein not being expressed at all. The “zero” expression may be achieved by any method known to one of skill in the art including RNA silencing, modification of the expression process (for example, disruption of the promoter function), or complete or partial removal (knock out) of the nucleic acid encoding the enzyme from the genome.

The terms nucleic acid “constructs” or “vectors” and like terms should be taken broadly to include any nucleic acid (including DNA and RNA) suitable for use as a vehicle to transfer genetic material into a cell. The terms should be taken to include plasmids, viruses (including bacteriophage), cosmids, and artificial chromosomes. Constructs or vectors may include one or more regulatory elements, an origin of replication, a multicloning site, and/or a selectable marker. The constructs or vectors may be adapted to allow expression of one or more genes encoded by the construct or vector. Nucleic acid constructs or vectors include naked nucleic acids as well as nucleic acids formulated with one or more agents to facilitate delivery to a cell (e.g., liposome-conjugated nucleic acids).

Exogenous nucleic acids may be introduced to the bacterium using any method known in the art. For example, transformation (including transduction or transfection) may be achieved by electroporation, ultrasonication, polyethylene glycol-mediated transformation, chemical or natural competence, protoplast transformation, prophage induction or conjugation (see, e.g., Sambrook, Molecular Cloning: A Laboratory Manual, Cold Spring Harbor Laboratory Press, Cold Spring Harbor, 1989).

The use of electroporation has been reported for several carboxydotrophic acetogens, including C. ljungdahlii (Kopke, PNAS, 107:13087-13092, 2010; WO/2012/053905), C. autoethanogenum (WO/2012/053905), C. aceticum (Schiel-Bengelsdorf, Synthetic Biol, 15: 2191-2198, 2012), and A. woodii (Strätz, Appl Environ Microbiol, 60: 1033-1037, 1994). The use of electroporation has also been reported in Clostridia, including C. acetobutylicum (Mermelstein, Biotechnol, 10: 190-195, 1992), and C. cellulolyticum (Jennert, Microbiol, 146: 3071-3080, 2000). Additionally, prophage induction has been demonstrated for carboxydotrophic acetogens, including C. scatologenes (Parthasarathy, Development of a Genetic Modification System in Clostridium scatologenes ATCC 25775 for Generation of Mutants, Masters Project, Western Kentucky University, 2010), and conjugation has been described for many Clostridia, including C. difficile (Herbert, FEMS Microbiol Lett, 229: 103-110, 2003) and C. acetobuylicum (Williams, J Gen Microbiol, 136: 819-826, 1990). Similar methods could be used in carboxydotrophic acetogens.

Methylation of the exogenous nucleic acid may be required if certain restriction systems are active in the host microorganism.

A shuttle microorganism may be a microorganism in which a methyltransferase enzyme is expressed, and is distinct from the destination microorganism. A shuttle microorganism can be used to introduce post-synthetic modifications to genetically engineered nucleic acids, which will protect the genetically engineered nucleic acids in the ultimate host organism. A destination microorganism, conversely, is a microorganism in which the genes included on an expression construct/vector are expressed and is distinct from the shuttle microorganism.

The bacterium may be any recombinant, carboxydotrophic, acetogenic bacterium. “Recombinant” refers to a microorganism that has been genetically modified from a wild-type or parent microorganism. “Carboxydotroph” refers to a microorganism, preferably a bacterium, which can tolerate a high concentration of carbon monoxide. “Acetogen” refers to a microorganism, preferably a bacterium, which naturally generates acetate/acetic acid as a product of anaerobic fermentation.

In particular, the bacterium may belong to the genus Clostridium. For example, the bacterium may be Clostridium aceticum, Clostridium acetobutylicum, Clostridium autoethanogenum, Clostridium beijerinckii, Clostridium butyricum, Clostridium carboxidovirans, Clostridium cellulolyticum, Clostridium cellulovorans, Clostridium difficile, Clostridium diolis, Clostridium drakei, Clostridium formicoaceticum, Clostridium kluyveri, Clostridium ljungdahlii, Clostridium magnum, Clostridium novyi, Clostridium pasterianium, Clostridium phytofermentans, Clostridium ragsdalei, Clostridium saccharbutyricum, Clostridium saccharoperbutylacetonicum, Clostridium scatologenes, Clostridium thermocellum, or a bacterium derived therefrom. The bacterium may be an acetogenic, carboxydotrophic bacterium. In a preferred embodiment, the bacterium is derived from Clostridium autoethanogenum, Clostridium ljungdahlii, or Clostridium ragsdalei. The bacterium may be derived from Clostridium autoethanogenum DSM10061, Clostridium autoethanogenum DSM23693 (LZ1561, a derivate of Clostridium autoethanogenum DSM10061), or Clostridium ljungdahlii DSM13528. In a preferred embodiment, the bacterium is Clostridium autoethanogenum DSM23693 comprising a gene encoding an secondary alcohol dehydrogenase comprising an inactivating mutation.

Other bacteria may also be used. For example, the bacterium may belong to the genus Acetobacterium, Alkaligenes, Bacillus, Brevibacterium, Candida, Corynebacterium, Enterococcus, Escherichia, Hansenula, Klebsiella, Lactobacillus, Lactococcus, Paenibacillus, Pichia, Pseudomonas, Ralstonia, Rhodococcus, Saccharomyces, Salmonella, Trichoderma, or Zymomonas. In particular, the bacterium may be Acetobacterium woodii, Alkalibaculum bacchii, Bacillus licheniformis, Bacillus subtilis, Blautia producta, Butyribacterium methylotrophicum, Corynebacterium glutamicum, Escherichia coli, Eubacterium limosum, Klebsiella oxytoca, Klebsiella pneumonia, Lactobacillus plantarum. Lactococcus lactis, Moorella thermautotrophica, Moorella thermoacetica, Oxobacter pfennigii, Pseudomonas putida, Ralstonia eutropha, Saccharomyces cerevisiae, Sporomusa ovata, Sporomusa silvacetica, Sporomusa sphaeroides, Thermoanaerobacter kiuv, Trichoderma reesei, Zymomonas mobilis, or a bacterium derived therefrom.

With respect to microorganisms (e.g., a bacterium), the term “derived” refers to the modification of a parent or wild-type microorganism. A bacterium comprising a gene encoding a secondary alcohol dehydrogenase comprising an inactivating mutation, may be derived from a parent or wild-type bacterium comprising a gene encoding a secondary alcohol dehydrogenase that does not comprise an inactivating mutation, or a parent or wild-type bacterium that does not comprise a gene encoding a secondary alcohol dehydrogenase. The bacterium may be derived from a parent or wild-type bacterium using any method known in the art, such as artificial or natural selection, mutation, or genetic recombination. The parent microorganism may be an organism that has been previously modified, but does not express or over-express one or more enzymes of the present invention (e.g., LZ1561). In a preferred embodiment, the bacterium is derived from Clostridium autoethanogenum, Clostridium ljungdahlii, or Clostridium ragsdalei. In a particularly preferred embodiment, the bacterium is derived from the bacterium deposited under DSMZ accession no. DSM23693 (i.e., LZ1561).

The bacterium may be isolated. For example, the bacterium may be physically isolated from its natural environment, from a culture comprising a two or more microorganisms, or from a culture comprising a heterogenous (genetically nonidentical) population of a single microorganism. A colony or culture grown from a single bacterium is also considered to be isolated.

The bacterium may be further modified to express or overexpress one or more enzymes in a biosynthetic pathway for making carbonyl-containing compounds, such as aldehydes or ketones. The biosynthetic pathways may be endogenous, partially endogenous, or fully exogenous to the bacterium. The biosynthetic pathways may comprise, for example, an exogenous enzyme or enzymes derived from other species or genera. In some cases, the exogenous enzymes may work in concert with endogenous enzymes to form a biosynthetic pathway that is naturally not present in that bacterium. For example, the production of acetone has been demonstrated in microorganisms that do not naturally produce acetone (WO/2012/115527). Genes encoding enzymes such as thiolase, coA-transferase, and acetoacetate decarboxylase may be added to a bacterium to enable the bacterium to make acetone. Additionally, or alternatively, an exogenous propanediol dehydratase may be added to a bacterium to enable the bacterium to convert meso-2,3-butanediol to 2-butanone (MEK). The exogenous propanediol dehydratase may be derived from Klebsiella oxtoca.

Gas fermentation is a metabolic process by which a gaseous substrate is used as a carbon and/or energy source for the production of ethanol or other products or chemicals. As used herein, the term “fermentation” encompasses both the growth phase and the product biosynthesis phase of the process. The gas fermentation is performed by a microorganism, typically bacteria.

The bacterial culture may be grown in any liquid nutrient medium that provides sufficient resources to the culture. The liquid nutrient medium may contain, for example, vitamins, minerals, and water. Examples of suitable liquid nutrient media are known in the art, including anaerobic media suitable for the fermentation of ethanol or other products (see, e.g., U.S. Pat. No. 5,173,429, U.S. Pat. No. 5,593,886, and WO 2002/08438).

The bacterial culture may be contained in a reactor (bioreactor). The reactor may be any fermentation device having one or more vessels and/or towers or piping arrangements for the growth of a bacterial culture. The reactor may be, for example, an immobilised cell reactor, a gas-lift reactor, a bubble column reactor (BCR), a circulated loop reactor, a membrane reactor, such as a hollow fibre membrane bioreactor (HFM BR), a continuous flow stirred-tank reactor (CSTR), or a trickle bed reactor (TBR). The reactor is preferably adapted to receive a gaseous substrate comprising CO, CO₂, and/or H₂. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

The fermentation should desirably be carried out under appropriate fermentation conditions for the production of carbonyl-containing compounds to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

The terms “increasing the efficiency,” “increased efficiency,” and the like, when used in relation to a fermentation process, include, but are not limited to, increasing one or more of the rate of growth of microorganisms catalyzing the fermentation, the growth and/or product production rate at elevated product concentrations, the volume of desired product produced per volume of substrate consumed, the rate of production or level of production of the desired product, and the relative proportion of the desired product produced compared with other by-products of the fermentation.

The gaseous substrate (“gas” or “feed gas” or “fermentation gas” or “substrate”) may be any gas which contains a compound or element used by a microorganism as a carbon and/or energy source in fermentation. The gaseous substrate will typically contain a significant proportion of H₂, CO₂, and/or CO, and may additionally contain N₂ or other gasses. In one embodiment, the gaseous substrate comprises CO. In another embodiment, the gaseous substrate comprises H₂, CO₂, and CO. In a further embodiment, the gaseous substrate is free or substantially free of O₂.

The exact composition of the gaseous substrate may vary. The gaseous substrate may contain, for example, about 20% to about 100% CO by volume, about 20% to 70% CO by volume, about 30% to 60% CO by volume, or about 40% to 55% CO by volume. In particular, the gaseous substrate may contain about 25%, about 30%, about 35%, about 40%, about 45%, about 50% CO, about 55% CO, or about 60% CO by volume. The gaseous substrate may contain, for example, about 1% to 80% CO₂ by volume or about 1% to 30% CO₂ by volume. The gaseous substrate may contain, for example, less than about 20% CO₂, less than about 15% CO₂, less than about 10% CO₂, less than about 5% CO₂, or about 0% (substantially no) CO₂ by volume. While it is not required for the gaseous substrate to contain H₂, the presence of H₂ may result in improved overall efficiency of desired product production. The gaseous substrate may comprise, for example, a 2:1, 1:1, or 1:2 ratio of H₂:CO. The gaseous substrate may comprise, for example, less than about 30% H₂, less than about 20% H₂, less than about 15% H₂, or less than about 10% H₂ by volume. In other embodiments, the gaseous substrate may comprise low concentrations of H₂, for example, less than about 5% H₂, less than about 4% H₂, less than about 3% H₂, less than about 2% H₂, less than about 1% H₂, or about 0% (substantially no) H₂ by volume.

In addition, it is often desirable to increase the CO concentration of a substrate stream (or CO partial pressure in a gaseous substrate) to increase the efficiency of fermentation reactions where CO is a substrate. Operating at increased pressures allows a significant increase in the rate of CO transfer from the gas phase to the liquid phase where it can be taken up by a microorganism. This, in turn, reduces the retention time, defined as the liquid volume in the bioreactor divided by the input gas flow rate. The optimum reaction conditions will depend partly on the particular microorganism used. However, in general, it is preferable to perform the fermentation at pressure higher than ambient pressure. Also, since a given CO conversion rate is, in part, a function of the substrate retention time and achieving a desired retention time dictates the required volume of a bioreactor, the use of pressurized systems can greatly reduce the volume of the bioreactor required and, consequently, the capital cost of the fermentation equipment. According to examples given in U.S. Pat. No. 5,593,886, reactor volume can be reduced in linear proportion to increases in reactor operating pressure. For example, bioreactors operated at 10 atm pressure need only be one tenth the volume of those operated at 1 atm pressure.

The composition of gas streams used to feed a fermentation reaction can have a significant impact on the efficiency and/or cost of the fermentation reaction. For example, the presence of O₂ may reduce the efficiency of an anaerobic fermentation process. Processing of unwanted or unnecessary gases in stages of a fermentation process before or after fermentation can increase the energy burden of stages. For instance, where the gas stream is compressed before entering a bioreactor, energy may be used to compress gases that are not needed in the fermentation.

The gaseous substrate may be sourced from an industrial process. In particular, the gaseous substrate may be a waste gas generated by an industrial process, such as ferrous metal product manufacturing (e.g., steel manufacturing), non-ferrous product manufacturing, petroleum refining, coal gasification, electric power production, carbon black production, ammonia production, methanol production, or coke manufacturing. In a preferred embodiment, the gaseous substrate is derived from a steel manufacturing gas.

The gaseous substrate may be sourced from the gasification of organic matter such as methane, ethane, propane, coal, natural gas, crude oil, low value residues from oil refinery (including petroleum coke or petcoke), solid municipal waste, or biomass. Biomass includes by-products obtained during the extraction and processing of foodstuffs, such as sugar from sugarcane, or starch from maize or grains, or non-food biomass waste generated by the forestry industry. Any of these carbonaceous materials may be gasified, i.e., partially combusted with oxygen, to produce synthesis gas (syngas). Syngas typically comprises mainly CO, H₂, and/or CO₂ and may additionally contain amounts of methane, ethylene, ethane, or other gasses. The operating conditions of the gasifier can be adjusted to provide a substrate stream with a desirable composition for fermentation or blending with one or more other streams to provide an optimised or desirable composition for increased alcohol productivity and/or overall carbon capture in a fermentation process.

It may be desirable to filter, scrub, or otherwise pre-treat the gaseous substrate before it is used in fermentation to remove chemical or physical impurities or contaminants. For example, source gasses may be passed through water or otherwise filtered to remove particulate matter, long chain hydrocarbons, or tars. However, such filtration or pre-treatment is not always required. It is sometimes possible to provide unfiltered, untreated gaseous substrate directly to the fermentation culture.

Although the gaseous substrate is typically provided in the form of a gas, the term “gaseous substrate” also encompasses substrates comprising CO, CO₂, and/or H₂ provided in alternate forms. For example, the gaseous substrate containing CO may be provided dissolved in a liquid. Essentially, a liquid may be saturated with a CO-containing gas and then added to a bioreactor. Exemplary methods include the use of a microbubble dispersion generator (Hensirisak, Appl Biochem Biotechnol, 101: 211-227, 2002) and the adsorption of a gaseous substrate onto a solid support.

The invention further provides methods of producing ketones, such as acetone or MEK, by culturing the bacterium comprising an inactivated secondary alcohol dehydrogenase encoded by a secondary alcohol dehydrogenase gene comprising an inactivating mutation, whereby the bacterium produces one or more of acetone and MEK. The method may further comprise recovering the acetone or MEK from the culture.

### EXAMPLES

The following examples further illustrate the invention but, of course, should not be construed to limit its scope in any way.

### Example 1

This example describes general materials and methods.

Microorganisms and Growth Conditions

C. autoethanogenum DSM10061 and DSM23693 (a derivate of DSM10061) and C. ljungdahlii DSM13528 were sourced from DSMZ (The German Collection of Microorganisms and Cell Cultures, Inhoffenstraβe 7 B, 38124 Braunschweig, Germany). C. ragsdalei ATCC BAA-622 was sourced from ATCC (American Type Culture Collection, Manassas, Va. 20108, USA) and E. coli DH5a was sourced from Invitrogen (Carlsbad, Calif. 92008, USA).

E. coli was grown under aerobic conditions at 37° C. in LB (Luria-Bertani) medium containing 10 g of tryptone, 5 g of yeast extract, and 10 g of NaCl per 1.0 L of media. Solid media contained 1.5% agar.

Clostridium strains were grown at 37° C. in PETC medium at pH 5.6 using standard anaerobic techniques (Hungate, In: Methods in Microbiology, 3B: 117-132, 1969; Wolfe, Adv Microb Physiol, 6: 107-146, 1971). Fructose (heterotrophic growth) or 30 psi CO-containing steel mill gas (collected from New Zealand Steel site in Glenbrook, NZ; composition: 44% CO, 32% N₂, 22% CO₂, 2% H₂) in the headspace (autotrophic growth) was used as substrate. For solid media, 1.2% Bacto agar (BD, Franklin Lakes, N.J. 07417, USA) was added.

### PETC Medium

| Component | Amount per L of medium | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution | 10 | ml |
| Wolfe's vitamin solution | 10 | ml |
| Yeast Extract (optional) | 1 | g |
| Resazurin (2 g/L stock) | 0.5 | ml |
| NaHCO₃ | 2 | g |
| Reducing agent | 0.006-0.008% | (v/v) |
| Fructose (for heterotrophic growth) | 5 | g |

### Trace Metal Solution

| Component | Amount per L of trace metal solution | |
|---|---|---|
| Nitrilotriacetic acid | 2 | g |
| MnSO₄•H₂O | 1 | g |
| Fe(SO₄)₂(NH₄)₂•6H₂O | 0.8 | g |
| CoCl₂•6H₂O | 0.2 | g |
| ZnSO₄•7H₂O | 0.2 | mg |
| CuCl₂•2H₂O | 0.02 | g |
| NaMoO₄•2H₂O | 0.02 | g |
| Na₂SeO₃ | 0.02 | g |
| NiCl₂•6H₂O | 0.02 | g |
| Na₂WO₄•2H₂O | 0.02 | g |

### Reducing Agent

| Component | Amount per 100 mL of reducing agent |
|---|---|
| NaOH | 0.9 g |
| Cysteine-HCl | 4 g |
| Na₂S | 4 g |

### Wolfe's Vitamin Solution

| Component | Amount per L of Wolfe's vitamin solution |
|---|---|
| Biotin | 2 mg |
| Folic acid | 2 mg |
| Pyridoxine hydrochloride | 10 mg |
| Thiamine HCl | 5 mg |
| Riboflavin | 5 mg |
| Nicotinic acid | 5 mg |
| Calcium D-(+)-pantothenate | 5 mg |
| Vitamin B12 | 0.1 mg |
| P-aminobenzoic acid | 5 mg |
| Thioctic acid | 5 mg |

Fermentations with C. autoethanogenum DSM23693 were carried out in 1.5 L bioreactors at 37° C. using CO-containing steel mill gas as sole energy and carbon source. A defined medium was prepared, containing: MgCl, CaCl₂ (0.5 mM), KCl (2 mM), H₃PO₄ (5 mM), Fe (100 μM), Ni, Zn (5 μM), Mn, B, W, Mo, Se (2 μM). The medium was transferred into the bioreactor and autoclaved at 121° C. for 45 minutes. After autoclaving, the medium was supplemented with thiamine, pantothenate (0.05 mg/L), biotin (0.02 mg/L) and reduced with 3 mM cysteine-HCl. The reactor vessel was sparged with nitrogen through a 0.2 μm filter to achieve anaerobic conditions. Prior to inoculation, the gas was switched to CO-containing steel mill gas, fed continuously to the reactor. The gas flow was initially set at 80 ml/min, and increased to 200 ml/min during mid-exponential phase, while the agitation was increased from 200 rpm to 350 rpm. Na₂S was dosed into the bioreactor at 0.25 ml/hr. Once the OD600 reached 0.5, the bioreactor was switched to a continuous mode at a rate of 1.0 ml/min (dilution rate 0.96 d-1). Samples were taken to measure the biomass and metabolites, and the in flowing and out flowing gasses were analysed.

### Metabolite Analysis

Gas composition of the headspace was measured on a Varian CP-4900 micro GC with two installed channels. Channel 1 was a 10 m Mol-sieve column running at 70° C., 200 kPa argon, and a backflush time of 4.2s, while channel 2 was a 10 m PPQ column running at 90° C., 150 kPa helium and no backflush. The injector temperature for both channels was 70° C. Run times were set to 120 s, but all peaks of interest would usually elute before 100 s.

HPLC analysis of metabolic end products was performed using an Agilent 1100 Series HPLC system equipped with a RID (Refractive Index Detector) operated at 35° C. and an Alltech IOA-2000 organic acid column (150×6.5 mm, particle size 5 μm) kept at 60° C. Slightly acidified water was used (0.005 M H₂SO₄) as mobile phase with a flow rate of 0.7 ml/min. To remove proteins and other cell residues, 400 μl samples were mixed with 100 μl of a 2% (w/v) 5-sulfosalicylic acid and centrifuged at 14,000×g for 3 min to separate precipitated residues. 10 μl of the supernatant was then injected into the HPLC for analysis.

GC analysis of metabolic end products was performed using an Agilent 6890N headspace GC equipped with a Supelco PDMS 100 1 cm fiber, an Alltech EC-1000 (30 m×0.25 mm×0.25 μm) column, and a flame ionization detector (FID). 5 ml samples were transferred into a Hungate tube, heated to 40° C. in a water bath, and exposed to the fiber for exactly 5 min. The injector was kept at 250° C. Helium was used as a carrier gas and was maintained at a constant flow rate of 1 ml/min. The oven was run at 40° C. for 5 min, followed by an increase of 10° C./min up to 200° C. The temperature was then further increased to 220° C. at a rate of 50° C./min followed by a 5 min hold at this temperature. Then, the temperature was decreased to 40° C. at a rate of 50° C./min followed by a 1 min hold at this temperature. The FID was kept at 250° C. with 40 ml/min hydrogen, 450 ml/min air, and 15 ml/min nitrogen as make up gas.

### Example 2

This example demonstrates identification of a secondary alcohol dehydrogenase responsible for reduction of acetone to isopropanol and MEK to 2-butanol in C. autoethanogenum, C. ljungdahlii, and C. ragsdalei.

Conversion of acetone to isopropanol has been described in carboxydotrophic bacteria such as C. autoethanogenum (WO 2012/115527), C. ljungdahlii (WO 2012/115527), and C. ragsdalei (WO 2012/115527) (Ramachandriya, Biotechnol Bioeng, 108: 2330-2338, 2011). A secondary alcohol dehydrogenase (ADH; EC 1.1.1.2) with similarity to an enzyme from C. beijerinckii (Ismaiel, J Bacteriol, 175: 5097-5105, 1993) was also identified (WO 2012/115527).

C. autoethanogenum comprises a secondary alcohol dehydrogenase having an amino acid sequence of SEQ ID NO: 1, encoded by a nucleic acid sequence of SEQ ID NO: 2. C. ljundahlii comprises a secondary alcohol dehydrogenase having an amino acid sequence of SEQ ID NO: 1 (NCBI YP 003780646.1), encoded by a nucleic acid sequence of a secondary alcohol dehydrogenase gene within the C. ljundahlii genome (NCBI NC_{—}014328.1:2755565..2756620, GeneID 9446102, locus tag CLJU-c24860). C. ragsdalei comprises a secondary alcohol dehydrogenase having an amino acid sequence of SEQ ID NO: 1, encoded by a nucleic acid sequence of SEQ ID NO: 3.

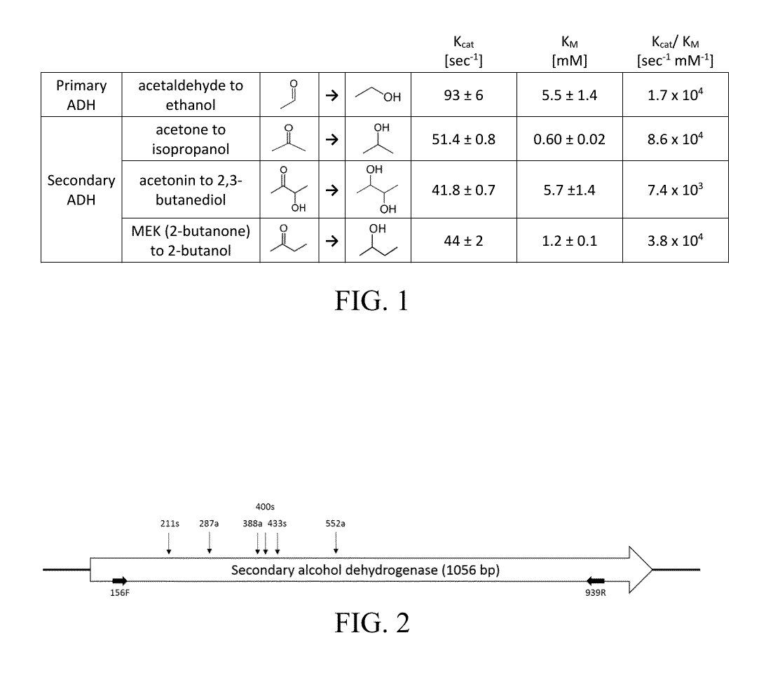

To demonstrate that this secondary alcohol dehydrogenase enzyme is responsible for the conversion of acetone to isopropanol, the secondary alcohol dehydrogenase of C. autoethanogenum was overproduced in E. coli and its activity was measured. SEQ ID NO: 2 was amplified from genomic DNA of C. autoethanogenum isolated as described in WO 2012/115527, cloned into a pBAD vector (Invitrogen) via KpnI and HindIII, and transformed into E. coli by electroporation using standard methods as described in Sambrook. The sequence of the ADH gene was verified by DNA sequencing. The transformed cells were grown in 100 mL LB with ampicillin at 37° C. until an OD600 of 0.8 was reached. At this point, 1 mL of 20% arabinose was added and the culture was incubated at 28° C. for the remainder of the expression. Cells were pelleted and the supernatant was decanted. Pellets were then resuspended in HEPES buffer (50 mM Na-HEPES and 0.2 mM DTT, pH 8.0), and 0.2 μL each of BENZONASE™ (Merck, 25 units/4) and lysozyme (Merck, 30 kU/μL) were added. The secondary alcohol dehydrogenase was then purified via the fused N-terminal His6tag using immobilized metal affinity chromatography. After a 30-min incubation on ice, the cells were lysed by sonication, the insoluble debris was pelleted, and the supernatant was clarified using a 0.2 micron filter. The clarified supernatant was added to TALON™ resin (Clontech) which had been thoroughly washed with lysis buffer. A bed volume of 500 μL was used for purification and the protein was allowed to bind to the TALON™ resin for an hour at 4° C. and then washed several times with lysis buffer. The protein was eluted from the column using lysis buffer supplemented with 150 mM imidazole, and the eluant was collected in 500 μL fractions. Aliquots taken through the purification process, as well as all elution fractions, were run on an SDS PAGE gel to confirm presence of the protein and determine the success of the purification. Proteins were exchanged into a storage buffer (50 mM potassium phosphate, 150 mM NaCl, 10% v/v glycerol, pH 7.0) using AMICON™ Ultra-4 Centrifugal Filter Units (Millipore). The protein was highly soluble when expressed in E. coli and could be readily purified. The activity assays were carried out in a UV/visspectrophotometer with quartz cuvettes using NADPH as a cofactor in a concentration of 0.2 mM and 3 mM MEK as a substrate. The assay mixture contained 50 mM Tris-HCl buffer (pH 7.5), with 1 mM DTT. The secondary alcohol dehydrogenase of C. autoethanogenum showed activity for reduction of acetone to isopropanol. In addition, activity for reduction of MEK to 2-butanol, acetoin to 2,3-butanediol, and acetaldehyde to ethanol was found. The highest activity was found with acetone, followed by MEK (FIG. 1).

In addition to secondary alcohol dehydrogenase, two butanol dehydrogenase (BDH) enzymes having activity on isopropanol have been described (Tan, J Basic Microbiol, 54: 996-1004, 2013). These butanol dehydrogenases are also present in C. autoethanogenum and C. ragsdalei.

### Example 3

This example demonstrates the inactivation of secondary alcohol dehydrogenase in C. autoethanogenum.

The identified secondary alcohol dehydrogenase of C. autoethanogenum (SEQ ID NO: 2) was inactivated using the ClosTron system (Heap, J Microbiol Meth, 70: 452-464, 2007). The intron design tool hosted on the ClosTron website was used to design a 344 bp targeting region (SEQ ID NO: 4), as well as identify six target sites (FIG. 2) on the sense and antisense strands. The targeting region was chemically synthesised in the vector pMTL007C-E2 containing a retro-transposition activated ermB marker (RAM).

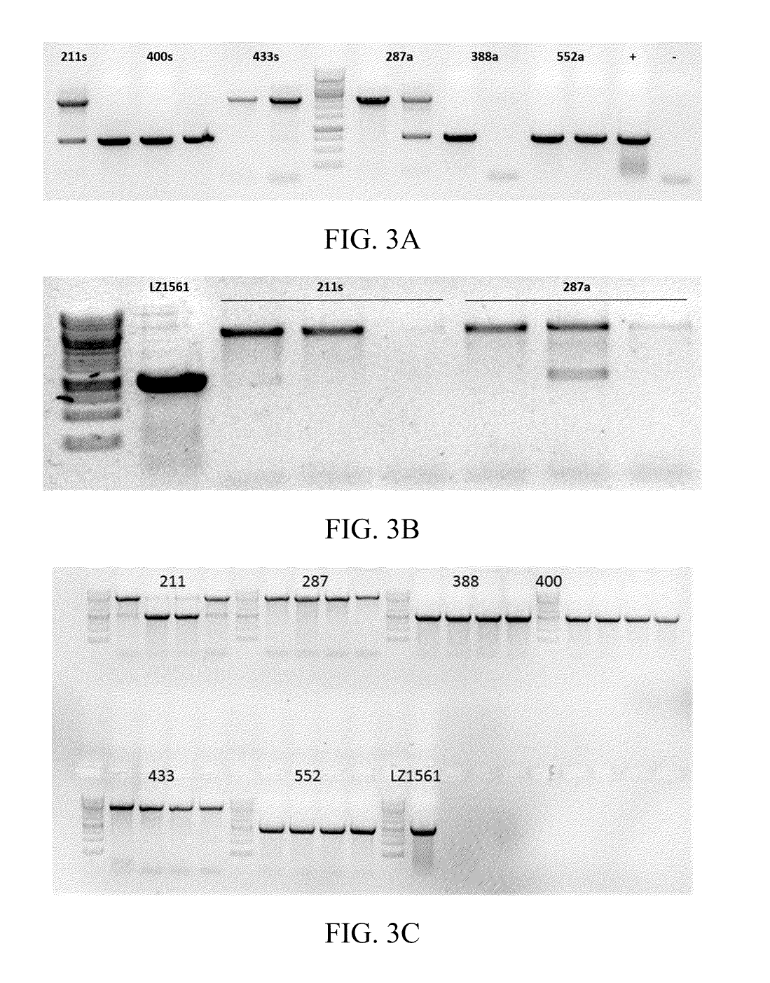

The vectors were introduced into C. autoethanogenum as described in WO 2012/053905. Single colonies grown on PETC MES with 15 μg/ml thiamphenicol were streaked on PETC MES with 5 μg/ml clarothromycin. Colonies from each target were randomly picked and screened for the insertion using flanking primers 155F (SEQ ID NO: 5), and 939R (SEQ ID NO: 6). Amplification was performed using the iNtron Maxime PCR premix. A PCR product of 783 bp indicated a LZ1561 genotype, while a product size of approximately 2.6 kb suggested the insertion of the group II intron in the target site (FIG. 3). The loss of plasmid was verified by PCR using primers with primers CatR (TTCGTTTACAAAACGGCAAATGTGA, SEQ ID NO: 7) and RepHF (AAGAAGGGCGTATATGAAAACTTGT, SEQ ID NO: 8) for amplification of the resistance marker (catP), and the gram positive origin of replication (pCB102).

The same strategy and plasmid can also be applied to C. ljungdahlii or C. ragsdalei. Transformation protocols have been previously described (WO 2012/053905) (Leang, Appl Environ Microbiol, 79: 1102-1109, 2013).

### Example 4

This example demonstrates elimination of acetone reduction to isopropanol in C. autoethanogenum having inactivated secondary alcohol dehydrogenase.

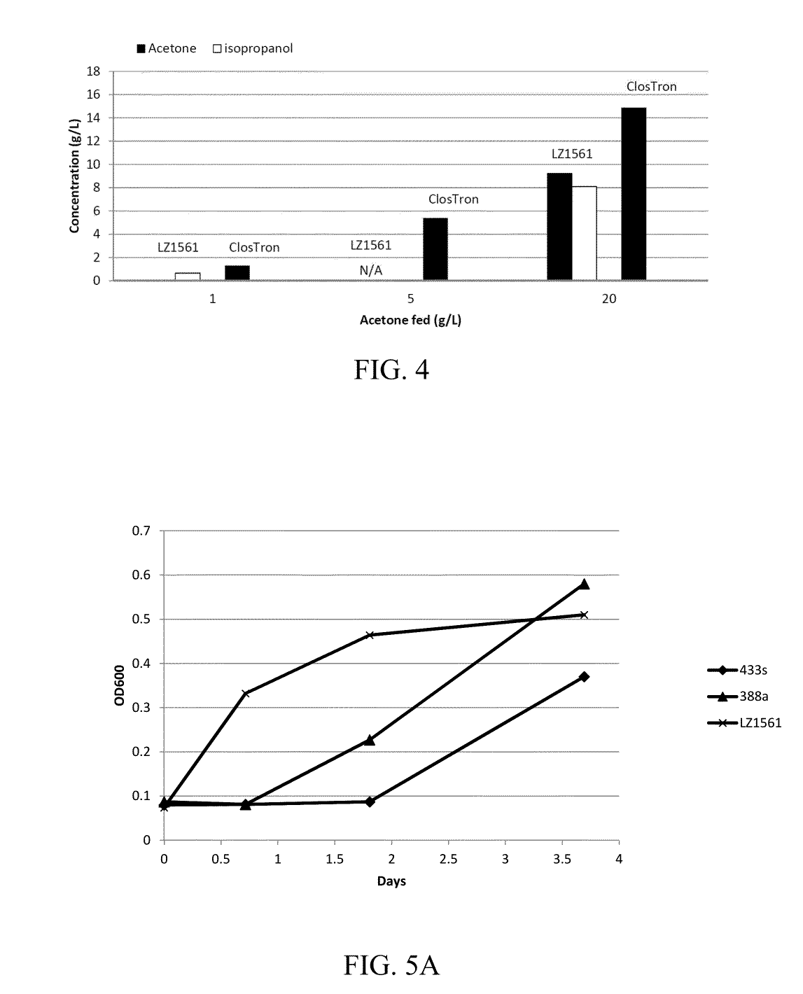

Acetone (0, 1, 5 and 20 g/L) was fed to serum bottle cultures of (1) LZ1561 and (2) ClosTron mutant C. autoethanogenum having inactivated secondary alcohol dehydrogenase with mutations at positions 287a and 211s. The LZ1561 strain fully converted 1 g/L of acetone into isopropanol, such that 0.65 g/L of isopropanol and no acetone was detected by HPLC. The final concentration of isopropanol in the serum bottles fed 20 g/L acetone was 8.1 g/L isopropanol, with 9.25 g/L acetone remaining No detectable conversion of acetone to isopropanol was observed in any of the ClosTron secondary alcohol dehydrogenase mutants (FIG. 4). Discrepancies between fed amount and measured amount were due to the difficulty of accurately adding acetone to a pressurized serum bottle.

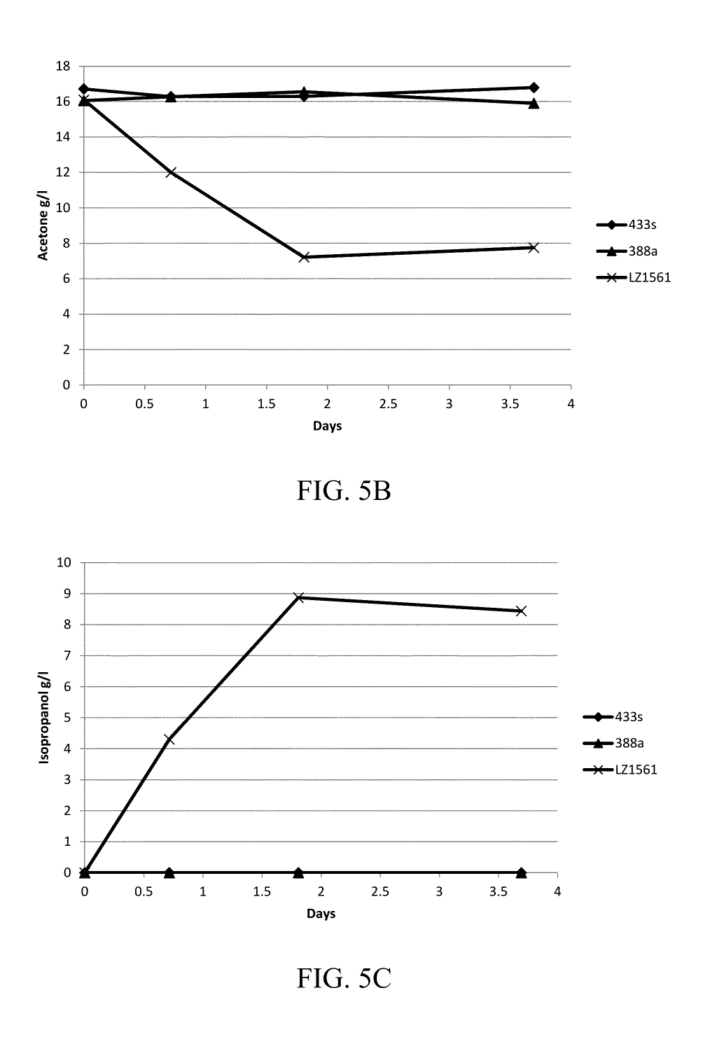

In an additional experiment, 16 g/L acetone was fed to serum bottle cultures of LZ1561 SecADH::erm 433s, LZ1561 SecADH::erm 388a, and LZ1561 in PETC MES. The cultures were grown under a mill gas headspace at 37° C. After 4 days of growth, LZ1561 had converted approximately half of the acetone into isopropanol. No isopropanol was detected in the secADH ClosTron mutants (FIG. 5).

Thus, while the LZ1561 strain very effectively reduced acetone to isopropanol, the mutant strains were unable to reduce acetone to isopropanol. This result was observed despite the presence of two butanol dehydrogenases, which apparently play no role in the reduction of acetone to isopropanol.

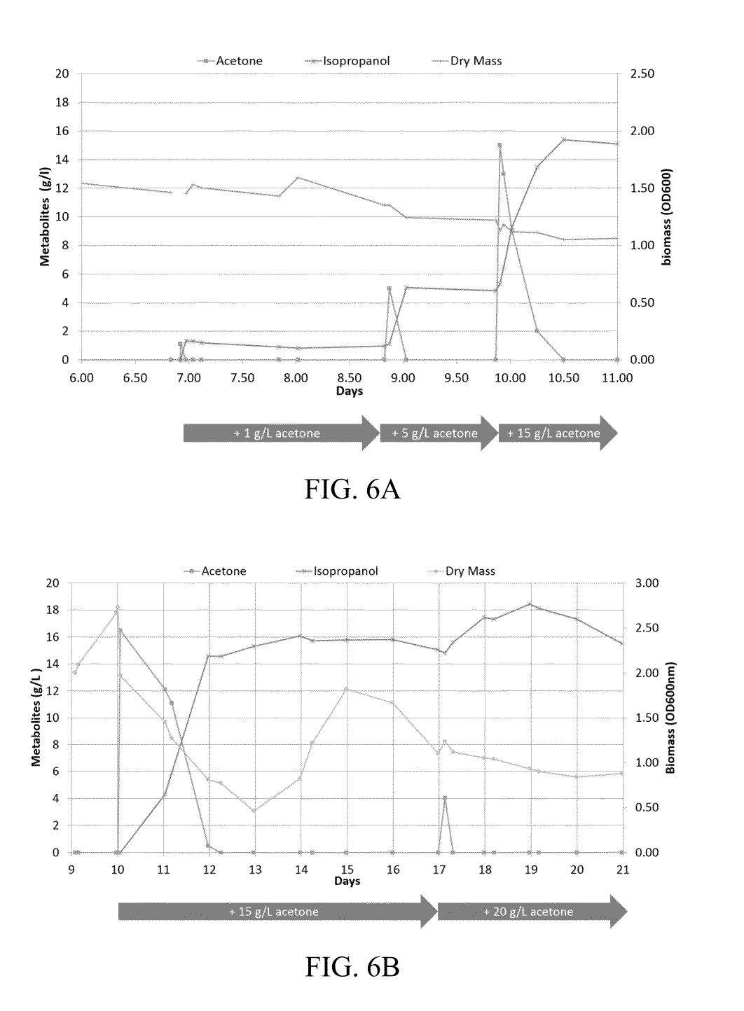

Experiments involving continuous culture in bioreactors were also performed. LZ1561 was grown in a bioreactor as described above. Once in continuous mode with stable biomass and metabolite production, acetone was added to both the bioreactor and the feed medium. Acetone was spiked into the reactor to a certain level, which was then obtained by continuous feeding. Initially, 1 g/L acetone was added. Once the metabolite concentrations stabilized, the concentration was increased to 5 g/L, 15 g/L, and—in a second experiment—to 20 g/L. Even at high concentrations of 15-20 g/L the culture converted all acetone to isopropanol at a high rate, demonstrating that the identified secondary alcohol dehydrogenase is highly effective (FIG. 6) (WO 2012/0252083).

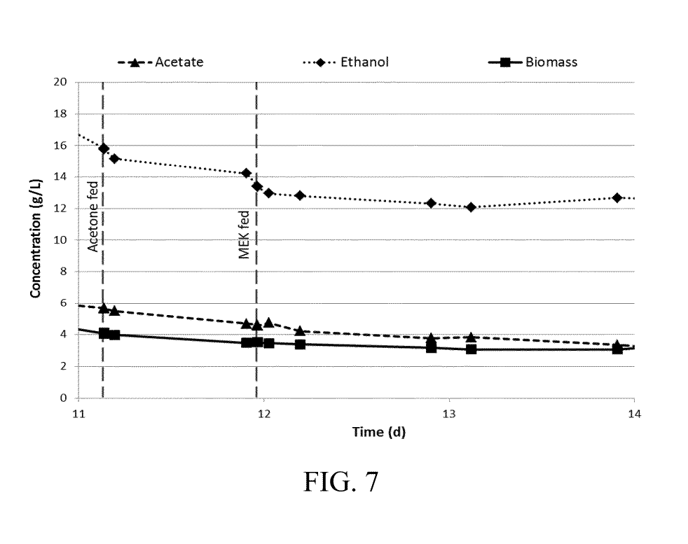

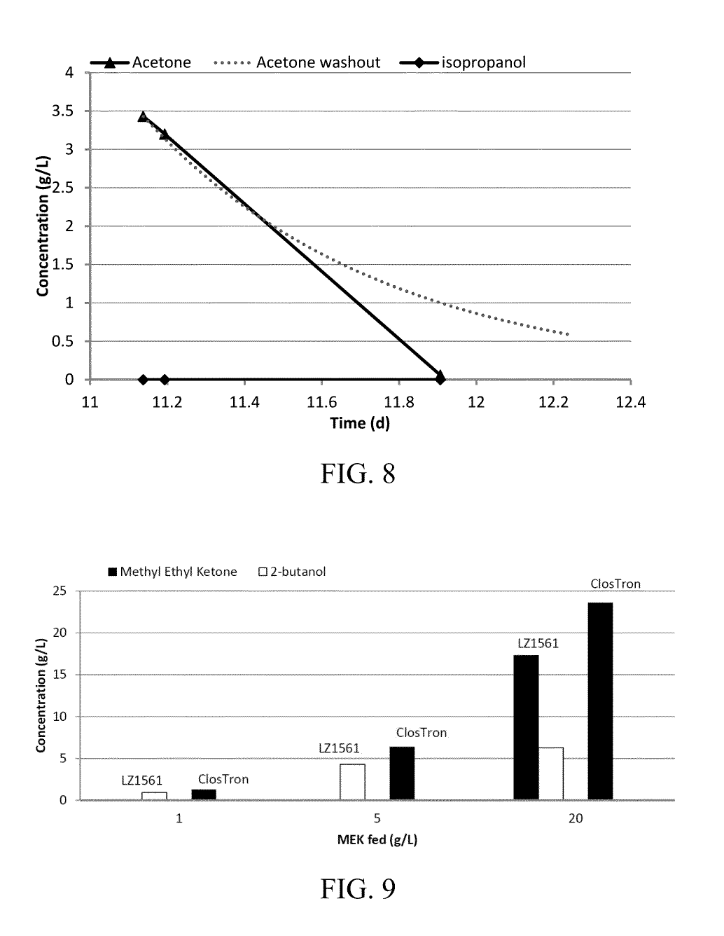

A similar bioreactor was experiment was performed with the ClosTron mutant having inactivated secondary alcohol dehydrogenase with mutations at position 211s. 5 g/L of acetone was spiked into the bioreactor. Unlike with LZ1561, where almost instant conversion of acetone to isopropanol was observed (FIG. 6), no conversion of acetone was observed in the ClosTron mutant (FIG. 7 and FIG. 8). Thus, conversion of acetone to isopropanol was completely eliminated in the Clos Tron mutant.

### Example 5

This example demonstrates acetone production from 1,2-propanediol without acetone reduction.

It has been previously demonstrated that C. autoethanogenum contains genes conferring the ability to stereospecifically dehydrate propane-1,2-diol to propanal and acetone when propane-1,2-diol is added to the medium. In LZ156, propanal and acetone are reduced to propan-1-ol and propan-2-ol. The acetone is reduced by the secondary alcohol dehydrogenase. When propane-1,2-diol is added to medium inoculated with a ClosTron mutant with inactivated secondary alcohol dehydrogenase, the acetone produced is not reduced to propan-2-ol.

Propane-1,2-diol was added to PETC-MES medium to 66 mM in serum bottles. The bottles were inoculated with C. autoethanogenum with ClosTron-inactivated secondary alcohol dehydrogenase (SecADH::ermB 287a) or LZ1561. After 96 hours of growth, LZ1561 produced 16.0±1.6 mM propan-2-ol, and 8.1±1.8 mM propan-1-ol, while the inactivated sADH strain produced 5.7±1.5 mM acetone, 6.0±1.8 mM propan-1-ol, and no detectable propan-2-ol.

### Example 6

This example demonstrates the production of acetone from CO in C. autoethanogenum with an inactivated secondary alcohol dehydrogenase gene

A plasmid with genes for synthesis of acetone including genes encoding thiolase, CoA-transferase and acetoacetate decarboxylase has been designed and introduced in C. autoethanogenum, resulting in isopropanol production with only little or no acetone (WO 2012/0252083). When such a plasmid was introduced in the ClosTron mutant of C. autoethanogenum with inactivated secondary alcohol dehydrogenase, acetone was produced with no isopropanol. Cultures were grown in 6-well plates at volumes of 6 mL per well, inside an anaerobic jar on CO/H2 gas at 25 psi. After 4 days growth the cultures produced an average of 0.95±0.01 g/L of acetone, 5.64±0.11 g/L ethanol, and 3.58±1.3 g/L of acetate. Due to the volatility of acetone at 37° C., the experiment was repeated with two additional 6-well plates with media, but no acetone producing bacteria. The acetone-producing cultures produced an average of 0.92±0.04 g/L acetone in 4 days. In addition, the background plates produced an average of 0.5±0.02 g/L acetone, which had been transferred via gas phase acetone. The final adjusted production was 1.9 g/L acetone taking into account the transfer of acetone.

Acetone production was also demonstrated in continuous culture in a bioreactor using the same strain and methods as described above. Over a 20 day period, approximate 0.2 g/L acetone was constantly observed in the broth (FIG. 11, wherein “acetone total” refers to the acetone in the medium (observed by HPLC) plus the acetone calculated to be removed from the medium by the gas passing through the fermenter). The bioreactor was running with a dilution rate of 1.5, giving a productivity of approximately 0.4 g/L/d in the broth. In addition to the concentration in the broth, a considerable amount of acetone (over 30% of the concentration in the broth) was stripped with the outcoming gas, as determined in a cooled knock-out pot of the outflowing gas. Given the volatility of acetone and the gas flow through the system, stripping of the acetone provides an ideal, low cost product separation, providing an advantage over isopropanol that is not as volatile and the majority needs to be extracted from the broth, for example through distillation. Stripping of the acetone also provides a driving force for product synthesis, avoiding end-product inhibition.

### Example 7

This example demonstrates the elimination of MEK to 2-butanol reduction.

MEK (0, 1, 5 and 20 g/L) was fed to serum bottle cultures of (1) LZ1561 and (2) ClosTron mutant C. autoethanogenum having inactivated secondary alcohol dehydrogenase with mutations at positions 287a and 211s. LZ1561 fully converted 1 and 5 g/L of MEK into 0.94 and 4.34 g/L of 2-butanol, respectively, with no detectable MEK remaining. The final concentration of 2-butanol in the serum bottles fed 20 g/L of MEK was 6.3 g/L 2-butanol, with 17.3 g/L of MEK remaining No conversion of MEK to 2-butanol was observed in any of the ClosTron secondary alcohol dehydrogenase mutants (FIG. 9).

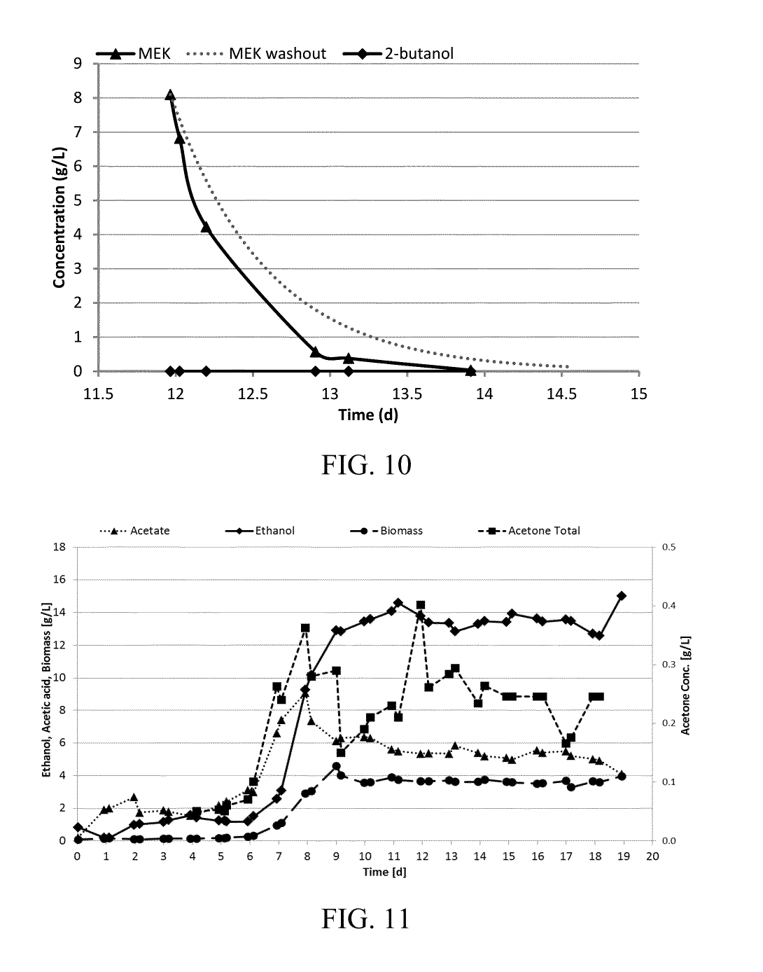

To further verify that inactivating the secondary alcohol dehydrogenase gene inactivated at position 211s completely eliminate the ability of C. autoethanogenum to convert MEK into 2-butanol 10 g/L of MEK was spiked into a bioreactor with the secondary alcohol dehydrogenase inactivated strain growing. No conversion of the MEK was observed (FIG. 7, FIG. 10).

### Example 8

This example describes the production of MEK from 2-3-butanediol in C. autoethanogenum with an inactivated secondary alcohol dehydrogenase gene.

It has been demonstrated that expressing propanediol dehydratase genes from K. oxytoca in C. autoethanogenum allows conversion of exogenous meso-2,3-butanediol to 2-butanone. The 2-butanone produced is reduced to 2-butanol by the secondary alcohol dehydrogenase present in C. autoethanogenum.

The ClosTron mutant of C. autoethanogenum with inactivated secondary alcohol dehydrogenase may is transformed with pMTL83155-pddABC, expressing the diol dehydratase from K. oxytoca, using established methods. The resultant strain is grown autotrophically in serum bottles in the presence of exogenous meso-2,3-butanediol. The butanediol converted to 2-butanone is not reduced to 2-butanol.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A recombinant, acetogenic, carboxydotrophic bacterium that lacks secondary alcohol dehydrogenase or comprises an inactivated secondary alcohol dehydrogenase.

2. The bacterium of claim 1, wherein the bacterium is a member of genus Clostridium.

3. The bacterium of claim 1, wherein the bacterium is derived from a bacterium selected from the group consisting of Clostridium autoethanogenum, Clostridium ljungdahlii, and Clostridium ragsdalei.

4. The bacterium of claim 3, wherein the Clostridium autoethanogenum is Clostridium autoethanogenum deposited under DSMZ Accession No. DSM23693.

5. The bacterium of claim 1, wherein the inactivated secondary alcohol dehydrogenase is inactivated primary-secondary alcohol dehydrogenase.

6. The bacterium of claim 1, wherein the inactivated secondary alcohol dehydrogenase is derived from a secondary alcohol dehydrogenase comprising the amino acid sequence of SEQ ID NO: 1.

7. The bacterium of claim 1, wherein the inactivated secondary alcohol dehydrogenase is encoded by a secondary alcohol dehydrogenase gene comprising an inactivating mutation.

8. The bacterium of claim 7, wherein the secondary alcohol dehydrogenase gene comprising the inactivating mutation is derived from the nucleic acid sequence of SEQ ID NO: 2 or SEQ ID NO: 3.

9. The bacterium of claim 7, wherein the inactivating mutation is an insertion.

10. The bacterium of claim 1, wherein the bacterium further comprises exogenous genes encoding one or more of a thiolase, a coA-transferase, and an acetoacetate decarboxylase.

11. The bacterium of claim 1, wherein the bacterium further comprises an exogenous gene encoding a propanediol dehydratase that converts meso-2,3-butanediol to MEK.

12. The bacterium of claim 11, wherein the propanediol dehydratase is a Klebsiella oxtoca propanediol dehydratase.

13. The bacterium of claim 1, wherein the bacterium produces one or more of acetone and MEK.

14. A method of producing acetone, comprising culturing the bacterium of claim 1, whereby the bacterium produces acetone.

15. The method of claim 14, further comprising recovering the acetone from the culture.

16. A method of producing MEK, comprising culturing the bacterium of claim 1, whereby the bacterium produces MEK.

17. The method of claim 16, further comprising recovering the MEK from the culture.


SEQUENCE LISTING (8 sequences)

SEQ ID NO: 1 - protein, 351 aa, Clostridium autoethanogenum
MKGFAMLGINKLGWIEKKNPVPGPYDAIVHPLAVSPCTSDIHTVFEGALGNRENMILGHEAVGEIAEVGSEVKDFKVGDRVIVPCTTPDWRSLEVQAGFQQHSNGMLAGWKFSNFKDGVFADYFHVNDADMNLAILPDEIPLESAVMMTDMMTTGFHGAELADIKMGSSVVVIGIGAVGLMGIAGSKLRGAGRIIGVGSRPVCVETAKFYGATDIVNYKNGDIVEQIMDLTHGKGVDRVIMAGGGAETLAQAVTMVKPGGVISNINYHGSGDTLPIPRVQWGCGMAHKTIRGGLCPGGRLRMEMLRDLVLYKRVDLSKLVTHVFDGAENIEKALLLMKNKPKDLIKSVVTF

SEQ ID NO: 2 - DNA, 1056 bp, Clostridium autoethanogenum
atgaaaggttttgcaatgttaggtattaacaaattaggatggattgaaaagaaaaacccagtgccaggtccttatgatgcgattgtacatcctctagctgtatccccatgtacatcagatatacatacggtttttgaaggagcacttggtaatagggaaaatatgattttaggccatgaagctgtaggtgaaatagccgaagttggcagcgaagttaaagattttaaagttggcgatagagttatcgtaccatgcacaacacctgactggagatctttagaagtccaagctggttttcagcagcattcaaacggtatgcttgcaggatggaagttttccaattttaaagacggtgtatttgcagattactttcatgtaaacgatgcagatatgaatcttgccatactcccagatgaaatacctttagaaagtgcagttatgatgacagacatgatgactactggttttcatggagcagaacttgcagacataaaaatgggctccagcgttgtagtaattggtataggagctgttggattaatgggaatagccggttccaaacttcgaggagcaggcagaattatcggtgttggaagcagacctgtttgtgttgaaacagctaaattttatggagcaactgatattgtaaattataaaaatggtgatatagttgaacaaatcatggacttaactcatggtaaaggtgtagaccgtgtaatcatggcaggcggtggtgctgaaacactagcacaagcagtaactatggttaaacctggcggcgtaatttctaacatcaactaccatggaagcggtgatactttaccaatacctcgtgttcaatggggctgcggcatggctcacaaaactataagaggaggattatgccccggcggacgtcttagaatggaaatgctaagagatcttgttctatataaacgtgttgatttgagtaaacttgttactcatgtatttgatggtgcagaaaatattgaaaaggcccttttgcttatgaaaaataagccaaaagatttaattaaatcagtagttacattctaa

SEQ ID NO: 3 - DNA, 1056 bp, Clostridium ragsdalei
atgaaaggttttgcaatgttaggtattaacaagttaggatggattgaaaagaaaaacccagtaccaggtccttatgatgcgattgtacatcctctagctgtatccccatgtacatcagatatacatacggtttttgaaggagcacttggtaatagggaaaatatgattttaggtcacgaagctgtaggtgaaatagctgaagttggcagtgaagttaaagattttaaagttggcgatagagttatcgtaccatgcacaacacctgactggagatccttagaagtccaagctggttttcaacagcattcaaacggtatgcttgcaggatggaagttttccaattttaaagacggtgtatttgcagattactttcatgtaaacgatgcagatatgaatcttgcaatacttccagatgaaatacctttagaaagtgcagttatgatgacagacatgatgactactggttttcatggggcagaacttgctgacataaaaatgggttccagtgttgtcgtaattggtataggagctgttggattaatgggaatagccggttccaaacttcgaggagcaggtagaattatcggtgttggaagcagacccgtttgtgttgaaacagctaaattttatggagcaactgatattgtaaattataaaaatggtgatatagttgaacaaataatggacttaactcatggtaaaggtgtagaccgtgtaatcatggcaggcggtggtgctgaaacactagcacaagcagtaactatggttaaacctggcggcgtaatttctaacatcaactaccatggaagcggtgatactttgccaatacctcgtgttcaatggggctgcggcatggctcacaaaactataagaggagggttatgtcccggcggacgtcttagaatggaaatgctaagagaccttgttctatataaacgtgttgatttgagcaaacttgttactcatgtatttgatggtgcagaaaatattgaaaaggcccttttgcttatgaaaaataagccaaaagatttaattaaatcagtagttacattctaa

SEQ ID NO: 4 - DNA, 344 bp, Artificial sequence
  Intron targeting region
aagcttataattatccttagatatcaatcttgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcgattatatctcgatagaggaaagtgtctgaaacctctagtacaaagaaaggtaagttagcaagattgacttatctgttatcaccacatttgtaca

SEQ ID NO: 5 - DNA, 20 bp, Artificial sequence
  156F primer
gccaggtccttatgatgcga

SEQ ID NO: 6 - DNA, 20 bp, Artificial sequence
  939R primer
gccccattgaacacgaggta

SEQ ID NO: 7 - DNA, 25 bp, Artificial sequence
  CatR primer
ttcgtttacaaaacggcaaatgtga

SEQ ID NO: 8 - DNA, 20 bp, Artificial sequence
  RepHF primer
gccccattgaacacgaggta